(19) United States
(12) Patent Application Publication
(10) Pub. No.: US 2024/0366289 A1
(43) Pub. Date: Nov. 7, 2024

(54) LEAKPROOF STRAW LID

(71) Applicant: Lifetime Brands, Inc., Garden City, NY (US)

(72) Inventors: Clay Burns, New York, NY (US); John Swartz, Ridgewood, NJ (US)

(21) Appl. No.: 18/775,186

(22) Filed: Jul. 17, 2024

Related U.S. Application Data
(63) Continuation of application No. 17/027,855, filed on Sep. 22, 2020, now Pat. No. 12,053,231.
(60) Provisional application No. 62/909,361, filed on Oct. 2, 2019.

(51) Int. Cl. A61B 18/14; A61B 18/00; G06N 3/047; G06N 3/08

(52) U.S. Cl. CPC A61B 18/1445; G06N 3/047; G06N 3/08; A61B 2018/00404; A61B 2018/0063; A61B 2018/00672; A61B 2018/00678; A61B 2018/00714; A61B 2018/00755; A61B 2018/00773

(57) ABSTRACT

A leakproof straw lid includes a lower lid and an upper lid. The lower lid is removably connected to a liquid containing vessel and includes a straw receiving hole for receiving a straw and a straw closing mechanism positioned adjacent the straw receiving hole. The upper lid is attachable to the lower lid and includes a straw receiving hole aligned with the straw receiving hole in the lower lid and at least one closing element configured to engage the straw closing mechanism in the lower lid. Rotation of the upper lid causes the at least one closing element to engage the straw closing mechanism which operates to compress or squeeze the straw and close the same.

### BACKGROUND

### Technical Field

The present invention relates to vessels capable of containing a liquid for drinking, like for example, tumblers, travel mugs and hydration bottles. More particularly, it relates to a leakproof straw lid for liquid containing vessels, such as tumblers, travel mugs and hydration bottles.

### Description of Related Art

The use of a straw for hydration bottles, travel mugs or tumblers is common. The term “vessel” as used herein is intended to refer to one or all types of such bottles, mugs or tumblers.

In some instances, the straws are inserted into the liquid containing portion of vessel when desired through a top or side opening and removed when not in use. In other instances, the lids to these vessels have lids where the straw access is a foldable piece integrated into the lid, where the folding opens and closes the access to the straw. In these versions, the mouth engaging part is a hard plastic and the flexible straw is often connected to the underside of the lid.

It would be desirable to provide a liquid containing vessel where the user can use a straw that passes through the lid, and which vessel can be opened and closed by opening and closing the straw internal to the lid, thus maintaining an aesthetically pleasing appearance while not having to remove the straw and/or fold the same out of the way.

### SUMMARY OF THE INVENTION

In accordance with one aspect of the invention, the leakproof straw lid includes a lower lid and an upper lid. The lower lid is removably attached to a vessel capable of holding a liquid and includes a hole for receiving a straw. A straw closing mechanism is positioned adjacent the hole. The upper lid is releasable attachable to the lower lid and also includes a straw receiving hole aligned with the hole in the lower lid. The upper lid includes closing elements that are configured to engage the straw closing mechanism in the lower lid such that rotation of the upper lid causes the straw closing mechanism to squeeze the straw and close the same.

In accordance with another aspect, the closing mechanism includes a first straw pincher adjacent the hole and a second straw pincher positioned adjacent the hole and opposite the first straw pincher.

According to yet another aspect, the first and second straw pinchers each includes a straw engaging surface such that straw engaging surface of each pincher is positioned opposite each other and contacts the straw and squeezes the same closed when the upper lid is rotated.

According to a further aspect, the first straw pincher is pivotally positioned adjacent the hole and includes a first raised portion. The second straw pincher is pivotally positioned adjacent the hole and includes a second raised portion. Rotation of the upper lid causes the closing elements to engage the first and second raised portions and thus pivotally move the first straw pincher and the second straw pincher to physically contact the straw and squeeze the straw closed.

According to a yet a further aspect, the upper lid includes a downwardly extending circular rim that is received into the lower lid when connecting the upper lid to the lower lid. The closing elements are disposed along an internal surface of the circular rim and extend radially inward from the same.

Other aspects and features of the present principles will become apparent from the following detailed description considered in conjunction with the accompanying drawings. It is to be understood, however, that the drawings are designed solely for purposes of illustration and not as a definition of the limits of the present principles, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings wherein like reference numerals denote similar components throughout the views:

FIG. 1 a perspective view of the leakproof straw lid according to an implementation of the invention;

FIG. 2 is an exploded perspective view of the leakproof straw lid and straw according to an implementation of the invention;

FIG. 3 is an underside view of the an upper part of the leakproof straw lid according to an implementation of the invention;

FIG. 4 is a top view of a lower part of the leakproof straw lid shown in an open position according to an implementation of the invention; and

FIG. 5 is a top view of the lower part of the leakproof straw lid shown in a closed position according to an implementation of the invention.

### DETAILED DESCRIPTION

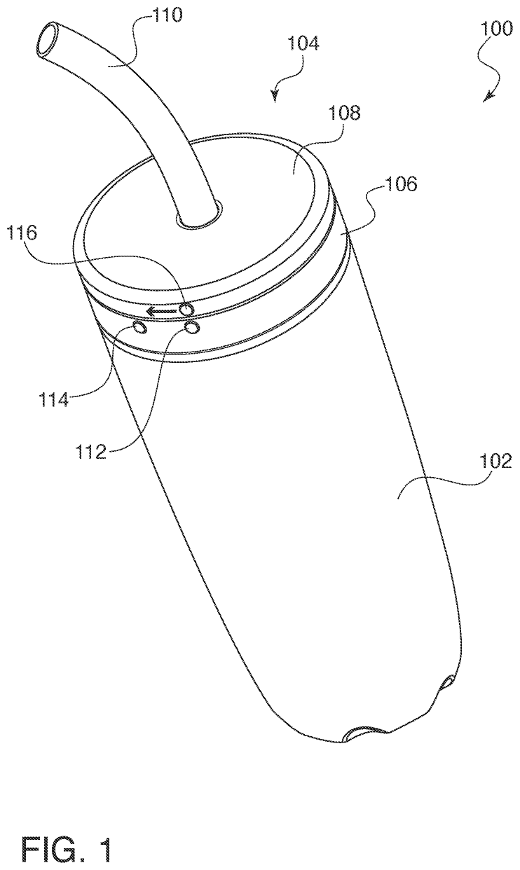

FIG. 1 shows an example of a drinking vessel such as, for example, a tumbler 100 according to an implementation of the invention. The tumbler 100 has a liquid containing body 102 and a lid 104 made up of lower lid 106 connected to the body 102, and an upper lid 108 connected to the lower lid 106. A straw 110 passes through both the upper lid 108 and lower lid 106 to access liquid contained in the body 102. As will be described in more detail below, the lower lid 106 has indicators 112 and 114 for indicating an open and closed position, respectively. The upper lid also includes a position indicator 116 that aligns with indicator 112 when in an open position and aligns with indicator 114 when rotated into a closed position.

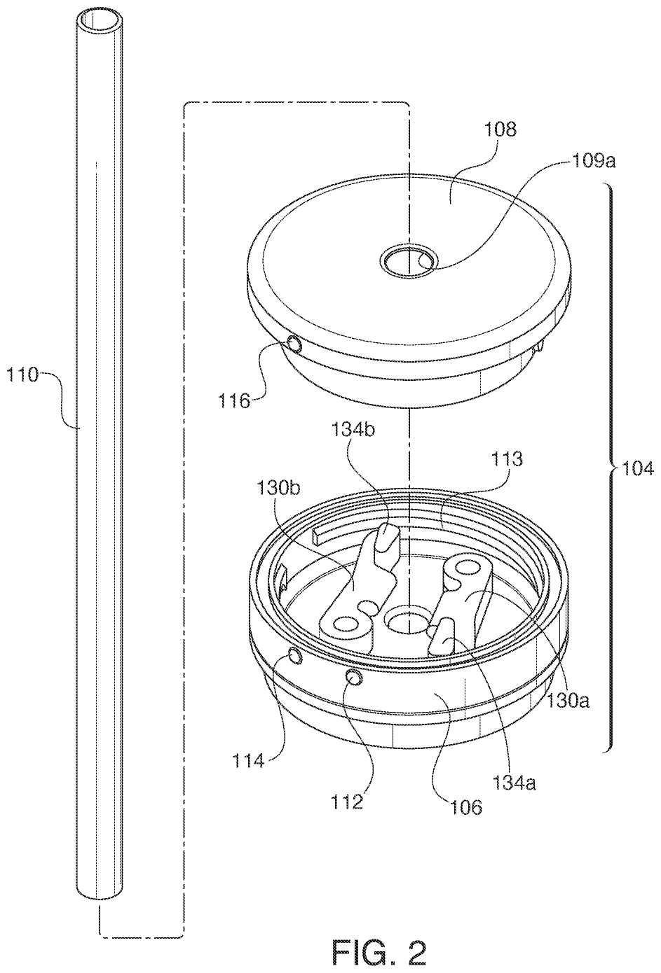

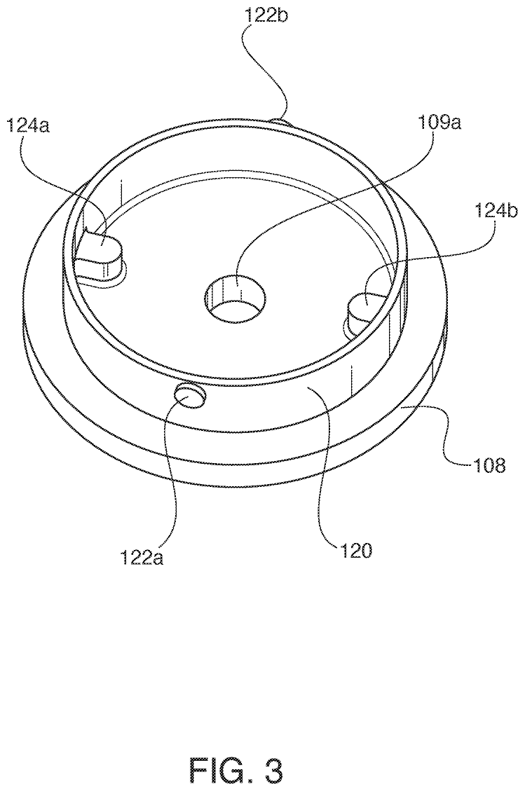

FIG. 2 shows the lid 104 with the straw 110 in an exploded view. Referring to both FIG. 2 and FIG. 3., the upper lid 108 has a hole 109a for receiving the straw 110 and includes downwardly extending circular rim 120 spaced from a circumferential edge. The circular rim 120 includes one or more exterior protrusions 122a and 122b (see FIG. 3) that are configured to engage the internal threading 113 of the lower lid 106 and function to secure the upper lid 108 to the lower lid 106 such that the upper lid 108 has limited rotating capability relative to the lower lid 106. In operation, when the upper lid 108 is connected to the lower lid 106, the position indicator 116 on the upper lid aligns with the “open” indicator 112 on the lower lid 106 (see FIG. 1). Internal protrusions 124a and 124b extend radially inward from the circular rim 120 and as will be shown and described below, operate to open and close the straw 110 when working in conjunction with the lower lid 106.

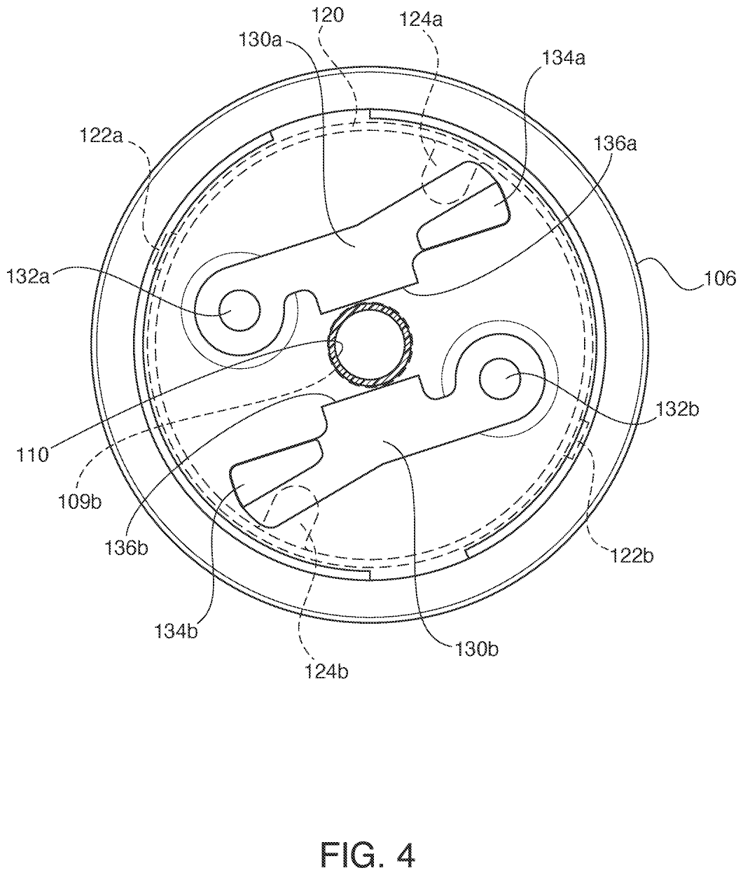

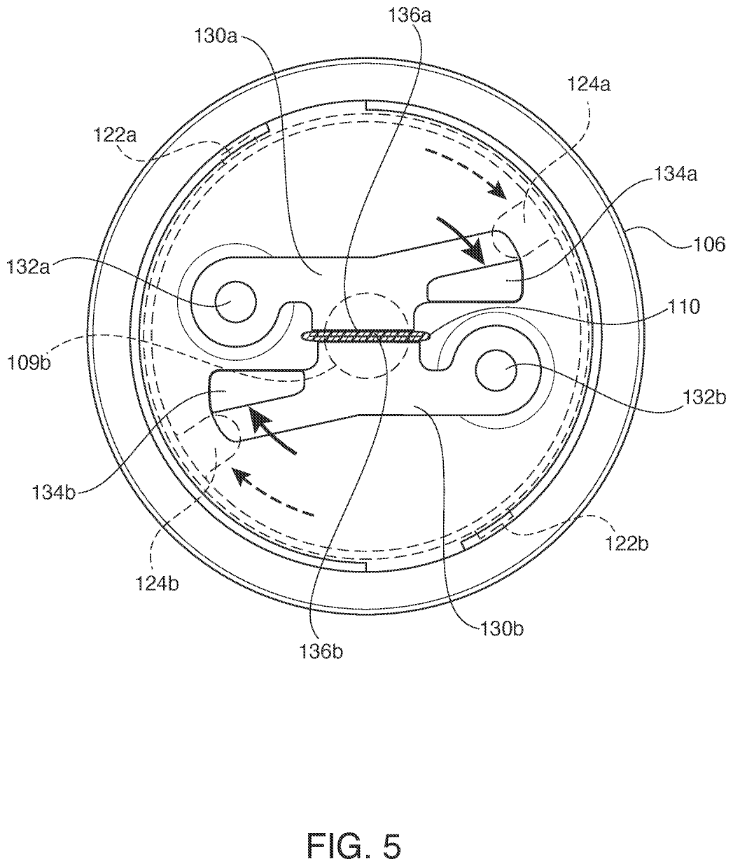

FIGS. 4 and 5 show the inside of the lower lid 106 with the circular rim 120 and internal protrusions 124a and 124b of the upper lid 108 shown in phantom. The lower lid 106 includes straw pinchers 130a and 130b that are pivotally or rotatably mounted above pivot points 132a and 132b, respectively. Straw pinchers 130a and 130b each include a raised portion 134a and 134b, respectively, at the distal end of the same relative to the pivot points 132a and 132b (See FIGS. 2, 4 and 5). Straw pinchers 130a and 130b each also include a straw engaging surface 136a and 136b, respectively, positioned opposite each other across the straw receiving opening 109b. FIG. 4 shows the straw pinchers 130a and 130b in their open position. In one implementation, this open position is the biased position for straw pinchers 130a and 130b such that the opening 109b in the lower lid 108 is open to receiving the straw 110. In one exemplary implementation, this biased open position can be achieved using springs in the pivot points of the pinchers 130a and 130b.

As will be appreciated by the present disclosure, in order to squeeze or pinch the straw 110 such that it is closed and does not leak, it is preferred that the squeezing or pinching is equally applied to opposing sides of the straw, and preferably with equal pressure from both sides.

It will be further appreciated that although the present invention is described in the context showing two pivotally mounted straw pinchers 130a and 130b, it is to be appreciated that in other implementations, one of the pinchers could be stationary or could be integrated into the lower lid as a stop or wall against which the one pincher would act to pinch or squeeze the straw closed when actuated by a closing element of the upper lid.

As can be seen in FIG. 4, the internal protrusions 124a and 124b from the circular rim 120 of the upper lid 108 are positioned immediately adjacent to and can be in slight contact with the raised portions 134a and 134b of the straw pinchers 130a and 130b, respectively. It will be appreciated that the internal protrusions 124a and 124b do not have to be in contact with the raised portions 134a and 134b when in the open position.

Referring to FIG. 5, when the upper lid 108 is rotated in the direction of the arrows (e.g., clockwise), the radial protrusions 124a and 124b engage and push against the raised portions 134a and 134b of the straw pinchers 130a and 130b, respectively. To close the straw, the upper lid 108 is rotated a small amount such that indicator 116 aligns with indicator 114 on the lower lid, and internally the straw engaging surfaces 136a and 136b come together and in doing so, tightly compress, squeeze or pinch the straw 110, thereby closing the same in a leakproof manner. In a preferred implementation, the straw 110 is made of food grade silicone or rubber. As used herein the terms squeeze, pinch and compress are interchangeable and clearly refer to the closing of the straw by pinching, squeezing or compressing the same.

As will be further appreciated, rotation of the upper lid 108 in the opposite direction (e.g., counterclockwise) will cause the release of the straw pinchers 130a and 130b, and alignment of the indicator 116 on the upper lid with the “open” indicator 112 on the lower lid, and thus opens the straw for drinking.

In accordance with other contemplated implementations, the mechanisms described above can be switched from the upper lid to the lower lid and from the lower lid to the upper lid, without departing from the intended scope of the present disclosure. By way of example, the straw pinchers 130 can be positioned in the upper lid, and the radial protrusions 124 can be positioned in the lower lid, such that rotation of the upper lid results in the same straw closing action as already described.

While there have been shown, described and pointed out fundamental novel features of the present principles, it will be understood that various omissions, substitutions and changes in the form and details of the methods described and devices illustrated, and in their operation, may be made by those skilled in the art without departing from the spirit of the same. For example, it is expressly intended that all combinations of those elements and/or method steps which perform substantially the same function in substantially the same way to achieve the same results are within the scope of the present principles. Moreover, it should be recognized that structures and/or elements and/or method steps shown and/or described in connection with any disclosed form or implementation of the present principles may be incorporated in any other disclosed, described or suggested form or implementation as a general matter of design choice. It is the intention, therefore, to be limited only as indicated by the scope of the claims appended hereto.

Having described preferred embodiments, which serve to illustrate various concepts, structures and techniques that are the subject of this patent, it will now become apparent to those of ordinary skill in the art that other embodiments incorporating these concepts, structures and techniques may be used. Additionally, elements of different embodiments described herein may be combined to form other embodiments not specifically set forth above.

What is claimed is:

1. A leakproof straw lid comprising:
a lower lid configured to be removably attached to a vessel capable of holding a liquid, the lower lid having a hole for receiving a straw and a straw closing mechanism positioned adjacent the hole; and
an upper lid releasably attachable to the lower lid, the upper lid having a straw receiving hole aligned with the hole in the lower lid and closing elements configured to engage the straw closing mechanism in the lower lid such that rotation of the upper lid relative to the lower lid causes the straw closing mechanism to squeeze the straw and close the same.

2. The leakproof straw lid according to claim 1, wherein the straw closing mechanism comprises:
a first straw pincher positioned adjacent the hole; and
a second straw pincher positioned adjacent the hole and opposite the first straw pincher.

3. The leakproof straw lid according to claim 1, wherein the straw closing mechanism comprises:
a first straw pincher pivotally positioned adjacent the hole; and
a second straw pincher pivotally positioned adjacent hole and opposite the first straw pincher, the first straw pincher and the second straw pincher each having straw engaging surfaces configured to physically contact the straw from opposing sides and squeeze the straw closed when the upper lid is rotated.

4. The leakproof straw lid according to claim 1, wherein the straw closing mechanism comprises:
a first straw pincher pivotally positioned adjacent the hole and having a first raised portion; and
a second straw pincher pivotally positioned adjacent hole and opposite the first straw pincher and having a second raised portion, wherein rotation of the upper lid causes the closing elements to engage the first raised portion and the second raised portion such that the first straw pincher and second straw pincher, respectively to physically contact the straw and squeeze the straw closed.

5. The leakproof straw lid according to claim 1, wherein the upper lid further comprises a downwardly extending circular rim that is received into the lower lid, the closing elements being disposed along an internal surface of the circular rim and extending radially inward from the circular rim.

6. The leakproof straw lid according to claim 5, further comprising at least one protrusion extending radially outward from an exterior surface of the circular rim, the at least one protrusion enabling the upper lid to be releasably connected to the lower lid.

7. The leakproof straw lid according to claim 1, further comprising an upper lid indicator and at least one indicator on the lower lid, wherein the upper lid indicator is configured to align with the at least one indicator on the lower lid to represent either an open or closed state of the straw.

8. The leakproof straw lid according to claim 1, wherein the straw comprises a food grade silicone.

9. A leakproof straw lid comprising:
a lower lid releasably attached to a vessel capable of holding a liquid, the lower lid having a first straw hole for receiving a straw and a straw closing mechanism positioned adjacent the first straw hole, the straw closing mechanism comprising at least one straw pincher positioned adjacent the first straw hole; and
an upper lid rotatably attached to the lower lid, the upper lid having a second straw receiving hole aligned with the first straw hole in the lower lid and at least one closing element configured to engage the at least one straw pincher in the lower lid such that rotation of the upper lid relative to the lower lid causes the at least one straw pincher to squeeze the straw and close the same.

10. The leakproof straw lid according to claim 9, wherein the upper lid further comprises a downwardly extending circular rim that is received into the lower lid, the at least one closing element being disposed along an internal surface of the circular rim and extending radially inward from the circular rim.

11. The leakproof straw lid according to claim 10, further comprising at least one protrusion extending radially outward from an exterior surface of the circular rim, the at least one protrusion enabling the upper lid to be releasably connected to the lower lid.

12. The leakproof straw lid according to claim 9, further comprising an upper lid indicator and at least one indicator on the lower lid, the upper lid indicator being configured to align with the at least one indicator on the lower lid to represent either an open or closed state of the straw.

13. The leakproof straw lid according to claim 9, wherein the straw comprises a food grade silicone.
